(19) United States
(12) Patent Application Publication
(10) Pub. No.: US 2014/0315289 A1
(43) Pub. Date: Oct. 23, 2014

(54) DISPOSABLE CARTRIDGE FOR ERYTHROCYTE MECHANICAL FRAGILITY TEST

(71) Applicant: Blaze Medical Devices, LLC, Ann Arbor, MI (US)

(72) Inventors: Michael Tarasev, Pinckney, MI (US); Kenneth Alfano, Canton, MI (US); Sumita Chakraborty, Ann Arbor, MI (US)

(21) Appl. No.: 14/320,344

(22) Filed: Jun. 30, 2014

Related U.S. Application Data
(62) Division of application No. 13/708,980, filed on Dec. 8, 2012.
Application No. 13/708,980 is a continuation-in-part of application No. 13/412,691, filed on Mar. 6, 2012, now abandoned.
Application No. 13/412,691 is a continuation-in-part of application No. 13/213,576, filed on Aug. 19, 2011, now abandoned.
Application No. 13/412,691 is a continuation of application No. 12/690,916, filed on Jan. 20, 2010, now Pat. No. 8,026,102.
Application No. 13/213,576 is a continuation-in-part of application No. 12/690,916, filed on Jan. 20, 2010, now Pat. No. 8,026,102.

(51) Int. Cl. G01N 33/49

(52) U.S. Cl. CPC G01N 33/49
USPC 435/288.7

(57) ABSTRACT

A disposable cartridge for use in red blood cell mechanical fragility profiling, comprising a stressing section with a lysing chamber in which the sample can receive multiple increments of mechanical stress to cause lysis, and a detection section with a transparent region that at least sometimes can have a thickness suitable to take optical readings that can directly or indirectly reflect a fraction/proportion of cells lysed after each increment of the stress, whereby said region can capture at least a representative portion of the sample that received each increment of stress. In some embodiments, the stressing section and the detection section are the same, and/or the transparent region is flexible or otherwise compressible, and/or the stress is ultrasonic. In some embodiments, the sections are separate, and/or the region is rigid or has a fixed thickness, and/or the stress may comprise cell-to-wall interaction and associated fluidic or viscous shear stress induced via concentric/coaxial objects.

### CROSS-REFERENCE TO RELATED APPLICATIONS

This application is a US divisional application claiming the benefit of Ser. No. 13/708,980, filed Dec. 8, 2012, which is a US continuation-in-part application claiming the benefit of Ser. No. 13/412,691, filed Mar. 6, 2012, and also of Ser. No. 13/213,576, filed Aug. 19, 2011, which are respectively a US continuation application and a US continuation-in-part application of US nonprovisional application 12/690,916, filed Jan. 20, 2010 (which has issued as U.S. Pat. No. 8,026,102 on Sep. 27, 2011). All of these are herein incorporated by reference in their entireties.

### TECHNICAL FIELD

This disclosure is in the fields of medical devices and basic or clinical laboratory research. More particularly, it is in the field of fragility-measuring tests for red blood cells, and more particularly such tests employing mechanical stress.

### BACKGROUND

This section contains general background material, which is not necessarily prior art.

Red blood cell (RBC, erythrocyte) membrane fragility can be measured in various ways, principally either osmotically or mechanically. In general, it involves subjecting a sample of cells to a stress and measuring how much hemolysis occurs as a result of the applied stress. In the case of mechanical fragility (MF), cell membranes are exposed to some kind of mechanical disturbance such as a shear stress—which may vary in intensity, duration, or other parameters—while the proportion of cells lysing is tracked. This enables cells' overall susceptibility to hemolysis to be characterized and presented (comprehensively or selectively) in various ways. Fragility indices or profiles (single-parameter or multi-parameter) for erythrocytes may be desirable for research purposes or for clinical purposes. Applications may include blood product quality testing, diagnostics, clinical research, or basic research.

### BRIEF SUMMARY OF THE INVENTION

This section briefly and non-exhaustively summarizes the subject matter of this disclosure. As this is a divisional filing, note throughout that certain portions of this overall disclosure may be more directly relevant than others (e.g. relative to the direct '980 parent). Also, some things are more emphasized and/or explicit.

Devices and methods for measuring red blood cell (RBC) fragility can be useful for characterizing blood product or patient blood, in a wide variety of possible applications. Fragility measurements involve some means for applying known sources/amounts of a stress, as well as some means for determining the extent of hemolysis resulting from particular extent(s) of the stress. There are different ways this extent-of-hemolysis can be measured (in conjunction with various means of applying the stress), including various types of spectral analysis as well as cell-counting.

This disclosure pertains to a general-purpose RBC mechanical fragility testing system utilizing a single-use disposable component for holding blood samples, with said component capable of serving in effect as both a stressing chamber and a detection chamber—albeit optionally in different portions of said component. In some embodiments, no fluidic transfer is needed within the disposable component between stressing and detection of portions of a sample. Employing a disposable/consumable cartridge or chip or other such piece for housing each sample to be tested can enable convenient testing of discrete samples with minimal cleaning or risk of contamination—among other benefits. Such single-use components can be configured to subdivide a given sample into multiple subsamples to facilitate concurrent stressing for a multi-dimensional profile, and/or be configured to receive multiple samples from multiple respective sources to facilitate multiplex testing.

This disclosure also addresses utilizing sonication to subject RBC to high-energy mechanical stress as part of a particular approach to measuring RBC mechanical fragility. “Low-energy” mechanical fragility, such as that utilizing a typical bead mill, tends to more directly reflect erythrocyte membrane properties (e.g. related to its integrity), whereas “high-energy” mechanical fragility, such as that utilizing a sonicator, tends to more directly reflect hemoglobin viscosity and cell size/volume. (Note that both general kinds of fragility assays could be useful for different purposes, and potentially could be used in conjunction.)

The scope of the invention is defined by the claims. A more complete understanding of embodiments on the present disclosure will be afforded to those skilled in the art, as well as the realization of additional advantages thereof, by consideration of the following detailed description of one or more embodiments. Reference will be made to the appended sheets of drawings that will first be described briefly.

### BRIEF DESCRIPTION OF THE DRAWINGS

This section briefly describes the accompanying drawings for this disclosure. All drawings are for illustrative and explanatory purposes only and not intended to limit the present invention to any example embodiments depicted herein.

FIG. 1 shows a single-sample disposable cartridge for testing RBC fragility via sonication, the cartridge being in effect both a stressing chamber and an optical cuvette.

FIG. 2 shows a single disposable sample cartridge being inserted into an ultrasonic RBC fragility test system wherein a sonication head is ready to be moved into place.

FIG. 3 shows beneath the device shroud of an ultrasonic RBC fragility test system, wherein a sonication mechanism, cooling and alignment fixture, light emitter and optical detector, and control board are visible.

FIG. 4 shows a complete ultrasonic RBC fragility test system, with supporting computer (for control and analysis). Note that this particular version holds only one cartridge at a time, and each cartridge holds only one sample (although multiple such systems could be run by the same computer concurrently)

FIG. 5 shows a “multi-lane” ultrasonic RBC fragility testing system, which holds multiple single-sample cartridges for testing.

FIG. 6 shows a multi-sample disposable cartridge for testing RBC fragility via sonication, each section of the cartridge being in effect both a stressing chamber and an optical cuvette for its respective sample.

FIG. 7 shows a single-sample disposable cartridge for testing RBC fragility via sonication, wherein the sample gets split into multiple sub-samples, each section of the cartridge being in effect both a stressing chamber and an optical cuvette for its respective sub-sample.

FIG. 8 shows a single-sample, multi-sub-sample disposable cartridge placed within a casing containing components for cooling, sonication, and optical detection.

FIG. 9 shows a tower stacking multiple single-sample, multi-sub-sample disposable cartridges as “drawers,” each such drawer containing components for cooling, sonication, and optical detection of its respective sample, so as to integrate ultrasonic RBC fragility testing of multiple samples and their respective subsamples.

FIG. 10 shows a single-sample disposable cartridge to contain sample content during concentric-cylinder based testing of RBC mechanical fragility, the cartridge comprising both a stressing chamber and an optical cuvette.

FIG. 11 shows a core view of a base unit of a concentric-cylinder based system for testing of RBC mechanical fragility that utilizes a disposable cartridge to contain sample content during testing, the base unit comprising both a stressing portion and an optical portion, and with a door closed over the optical portion.

FIG. 12 shows a see-through view of a concentric-cylinder based RBC mechanical fragility test system from the front, which incorporates a disposable cartridge to contain sample content during testing.

FIG. 13 shows a see-through view of a concentric-cylinder based RBC mechanical fragility test system from the top, which incorporates a disposable cartridge to contain sample content during testing.

FIG. 14 shows a close-up front view of the optical portion of a concentric-cylinder based mechanical fragility test system for red blood cells.

### DETAILED DESCRIPTION

This section contains descriptive content for this disclosure. It is also to be understood that the terminology and phraseology used herein is for explanatory and exemplary purposes and not intended to be limiting, as the scope of the present invention is defined ultimately by the claims herein (as amended).

Erythrocyte mechanical fragility testing combines controlled physical (and more specifically, mechanical) stressing of cells along with a measurement of how much hemolysis occurs during such stressing. Most expressed definitions pertain to a notion of propensity for or susceptibility to hemolysis under mechanical stress. A wide range of research and clinical applications are possible for such metrics. Other (i.e., “non-mechanical”) kinds of fragility generally focus on other physical stresses—such as osmotic or photonic stress. Mechanical fragility has the potential to more relevantly (for select purposes) reflect erythrocyte membrane fragility, as osmotic fragility for example significantly depends on non-mechanical aspects such as intracellular ion concentration and transmembrane water/ion rates (in addition to mechanical properties). Sometimes the nomenclature or classification can vary, especially at the boundaries, or in cases of combinations or overlap. Likewise, within mechanical stress types, precise sub-categorization can vary as well (e.g. exact definitional limits or intersections of shear, pressure, stretch/tension, etc.).

There are many ways to provide the mechanical stress in a fragility assay, as well as many ways to measure the resulting hemolysis. As described by Tarssanen (1976), approaches to providing mechanical stress can be grouped into two broad categories: “high-energy” mechanical stress, and “low-energy” mechanical stress. Low-energy mechanical fragility, such as for example that utilizing a bead mill for the stress, tends to more directly reflect erythrocyte membrane properties, whereas high-energy mechanical fragility, such as that for example utilizing sonication for the stress, tends to more directly reflect hemoglobin viscosity and cell size. (Note that both general kinds of fragility assays could be useful for different purposes, and potentially for overlapping applications, and both kinds of stress are still “high” compared to that typically used for “deformability” measurements—in the sense of being potentially lethal to RBC and thus useful to hemolyze at least some of the cells as is needed for any “fragility” assay).

Certain kinds of mechanical stress, such as viscous stress, can be high-energy or low-energy; also, a fragility profile for some stress mechanisms may reflect membrane properties more directly in the lower ranges but reflect surface-to-volume ratio and viscosity more directly in the higher ranges. The implications of these differing kinds of effects can be that fragility tests employing different stress means and/or energy levels can correlate differently to a given phenomenon; to some extent this can be true even among different forms of low-energy stresses (e.g., bead mill vs. capillary tube). Just as osmotic and mechanical fragility can sometimes have inverse correlations to certain phenomena, likewise so can high-energy and low-energy variations of mechanical fragility. Also, sometimes stressors can be combined, such as when evaluating changes in mechanical fragility under differing osmolarities; relatedly, temperature or pressure or other factors can in some cases be relevant stress conditions.

However the stress gets provided for a given fragility test, there are also many ways to in effect measure the hemolysis resulting from the stress. Whenever the particular advantages of cell-counting (discussed in the noted Ser. No. 13/213,576 application) are not needed, the preferred approach is to use the Blood Hemolysis Analyzer disclosed in U.S. Pat. No. 7,790,464, issued Sep. 7, 2010, incorporated herein by reference in its entirety, which notably avoids the need for any separation steps to separate liquid from solid sample components (such as for example by centrifugation), as its innovation included exploiting a difference in “apparent” spectral absorption between extracellular/cell-free hemoglobin (Hb) and intracellular Hb, which in turn can be utilized to infer what proportion of a given sample is hemolysed. That patent notes that the desired sample thickness for its measurement can depend upon hematocrit, but in some embodiments may be between 0.05-0.5 mm. In general, among all possible means for detecting/measuring hemolysis level, optical ones are preferred; among optical means, spectral ones are preferred; among spectral means, those utilizing absorbance are preferred. As determining fractional hemolysis requires knowing the original (pre-lysis) hematocrit or a reasonable proxy therefor such as total hemoglobin concentration, two possible approaches for obtaining this piece of information are as follows: 1) achieving 100% hemolysis in at least one subsample as part of the stressing step, and/or 2) employing additionally the spectral analysis of blood in the visible spectral range (which can provide total Hb, which is the combined intracellular and extra-cellular hemoglobin).

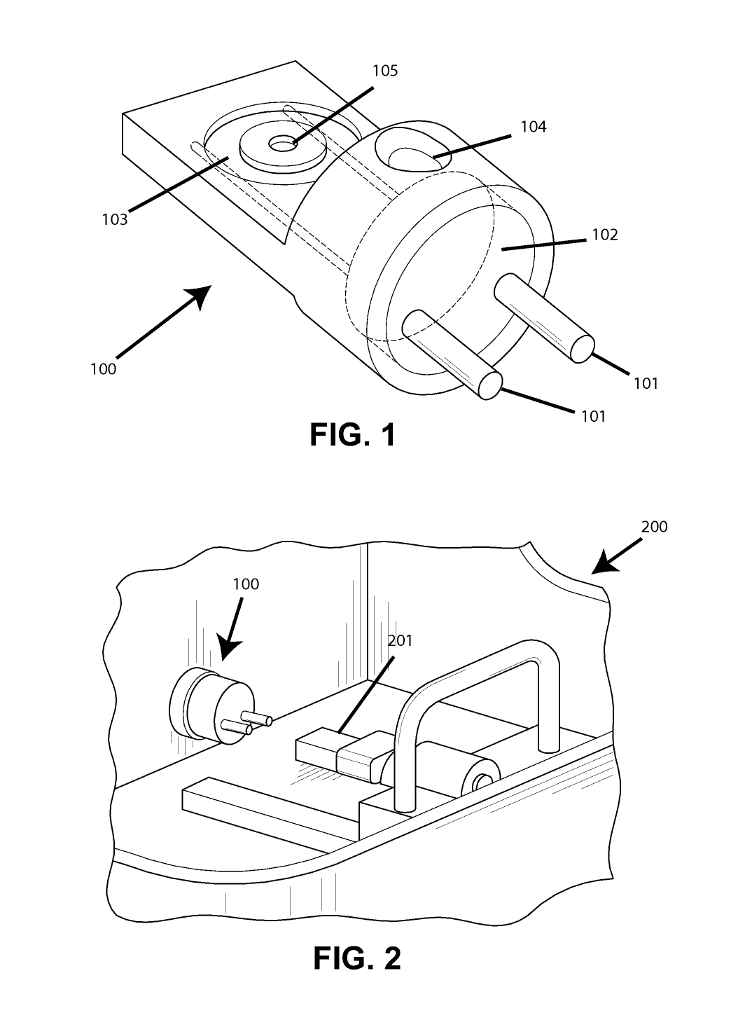

A fragility assay can involve outputting various kinds or amounts of fragility data—including specific values or indices, single-variable-parameter fragility profiles, and/or multi-variable-parameter (“multi-dimensional”) fragility profiles. (Note that terms such as “parameter” can be used to refer to either a stress parameter—which can be varied to create a profile—or a fragility index value which may itself be based on or derived from such a profile, depending on the context.) Data matrices comprising how much lysis occurs under various combinations of stress parameters can be used to yield profile-based parameters characterizing the sample tested (or the source it represents). Data of interest (or inferable information therefrom) could comprise how much lysis would be expected under a given set of stress parameters, what stress condition(s) would be expected to result in a given lysis level, or slopes or shapes of any such curves/trajectories—the latter of which can reveal subpopulations within a sample with their own discernible profiles. The distribution of cell ages (i.e., physiological/metabolic ages, distinguished from blood product/unit ages) within a given person or animal may be a factor in sub-populations within a sample exhibiting distinct characteristics; nevertheless, often it is desirable for a single value (e.g., an average via some fragility-based metric) to represent an overall sample. The particular fragility parameter(s) sought will likely depend on what is deemed most clinically or scientifically relevant for each particular application. Notably, a single-value index parameter can itself be determined from a profile, such as for example through an interpolation (e.g., to estimate from available data points how much stress duration at a given intensity of a given type would be required to lyse a given percent of RBC in a given sample/source). Such fragility parameter(s) can be computed using a processor, which could be any kind of computer or like unit capable of generating desired output from raw measurements. (Note that the original '102 patent's FIG. 1 shows total lysis, while its FIG. 6 caption cites induced lysis.)

Depending on the stress/lysis method employed for a given mechanical fragility (MF) testing approach, it may be important to dilute samples to ensure that all samples (or subsamples) have the same concentration of red cells (hematocrit) in order to have consistency in the rate/efficiency of hemolysis when subjected to stress. Later herein it is addressed the extent to which this is an issue for a sonication-based stress/lysis method. Also, in the case of red cell samples experiencing aggregation or coagulation, it can be useful to ascertain the role of such on the cells' susceptibility to induced hemolysis.

Sensitivity with regard to spectrally-based measures of hemolysis can be enhanced by accounting for multiple forms of hemoglobin—namely oxy, deoxy, meth, and/or carboxy. It's uncommon to determine the concentration of all four types, and perhaps unnecessary for the amount of precision typically needed (including for the present invention), but nevertheless remains an option. Other absorbent proteins that may potentially interfere with hemoglobin measurements can be accounted for with multi-wavelength analysis.

Clinical applications of fragility-based red cell metrics can include blood product quality testing by measuring sub-lethal cell damage (e.g., extent, rate, acceleration, trajectory, etc.) existing during storage any time after donation due to aging, processing, storage conditions, etc. Alternatively, fragility (osmotic and/or mechanical) can be useful in various patient diagnostics, including for example diagnosing conditions caused wholly or partially by drug and/or device based medical therapies—as well as any number of pathological diseases known or found to have an effect upon red cell membrane properties.

Regarding the blood-banking/transfusion-medicine (or TM) related uses, the related original application Ser. No. 12/690,916 described previously-unexplored approaches for using in vitro testing of RBC membrane properties to reflect blood quality loss in a time-independent manner; in particular, it suggested using RBC fragility, preferably mechanical, to indicate stored blood quality or loss thereof, and it notably presented preliminary in vitro data showing that RBC units of the same age can differ substantially in their mechanical fragility profiles. U.S. Pat. No. 8,263,408, issued Sep. 11, 2012 (resulting from a related divisional application of the noted parent) is also herein incorporated by reference and addresses “red blood cell suitability for transfusion,” tests for which can involve either donor (i.e. directly drawn from a donor or prospective donor) or donated (i.e. stored) red cells.

An extension of the blood product quality application can be to evaluate effects of blood product collection manner (e.g. apheresis vs. whole-blood) or manufacturing processes (e.g. leukoreduction, irradiation); or to validate associated processing equipment, as well as any storage/handling/transportation materials or conditions (e.g. bag material, storage or rejuvenation solutions, temperature ranges), by examining their respective effects upon blood quality or transfusion suitability (in terms of RBC fragility). Such RBC membrane properties can also be used to ascertain beforehand which units may be most amenable to certain manufacturing processes or storage conditions, by establishing a predictive correlation (directly or indirectly) between the in vitro property before subjection to said process or condition and relative in vivo performance. As with other applications, particular fragility assays or parameters may prove useful in combination with each other and/or other assays (e.g. biochemical).

Ongoing research in TM will progressively strengthen the case for clinical adoption of the MF test, with each round of studies likely enticing additional users for more use contexts (clinical opinion varies as to which kinds of studies would be most conclusive, as well as which applications would be most useful). Measurable degrees of quality/suitability could be used to reflect degrees of product acceptability, such as by selectively triaging those units deemed to be the “best” or least-degraded toward the most vulnerable patients (or for those unit transfusions where oxygenation is deemed the most critical, etc.). Moreover, the multi-parametric/multi-dimensional potential of MF could enable different “kinds” of quality to be discerned which may respectively prove more or less clinically relevant for different patients and/or patient groups/conditions. For example, post-transfusion in vivo RBC survival and RBC perfusion may each tend to be more associated with distinct respective multi-dimensional profile characteristics, as may resultant tissue oxygenation. Particular profile-based fragility parameter(s) or index(es) may thus prove indicative of RBC ability to adapt to the physical and/or chemical environment within the patient/recipient in a way that varies inter-donor, intra-donor, and even intra-unit (e.g., via sub-populations or cell-by-cell differences).

In effect, storage time has generally been the principal indicator of blood quality loss as applicable to particular individual units—although notably, the usefulness of even this indicator remains the subject of much controversy. This highlights the value of establishing a unit-specific RBC quality test to replace or supplement mere storage time as an indicator of storage lesion—as well as rates thereof, by testing at multiple points in time—thereby allowing modifications in today's inventory management practices (presently dominated by “first-in-first-out,” or FIFO). And in some cases, the RBC membrane properties of a patient's own blood may also be a factor in what kind of RBC properties are needed from stored blood they would receive in a transfusion. Variations among RBC units right at donation/collection can be at least partly attributable to “donor-to-donor” differences, which can also be reflected in subsequent degradation during storage (along with other factors). Dern et al. had first showed inter-donor variation in RBC “storageability” as measured by post-transfusion survival in vivo, and looked at some possible in vitro metrics (both membrane-related and strictly biochemical); Card et al. subsequently showed related findings (regarding inter-donor differences in certain RBC membrane properties). In some cases such differences may result from some identifiable medical condition, but there can also be a substantial range of variation among “healthy” donors. Also notable is that even “same-donor” differences can exist from one donation to the next.

In other (non-TM) applications, mechanical fragility (MF) can be used to evaluate or validate various blood-handling devices (or medical procedures employing them) by measuring sub-lethal RBC injury they cause manifesting in higher MF (e.g. Kamaneva et al. 2002, Yazer et al. 2008); relatedly, MF testing can be used to calibrate or account for MF to facilitate a consistent standard of evaluation of the actual hemolysis caused by blood-handling devices (e.g. Gu et al. 2005). Note also that evaluation of blood-handling devices via their effects on patients can be distinguished from actual patient diagnosis of a condition for which such a device may in fact be a cause.

There still as yet is no well-established or universally-standardized means to test for RBC MF—a fact that may have limited its utility for any of the above-noted or other applications. Of course, a vital aspect of optimal implementation for any application of any new life-science test includes the accumulation of copious output and associated correlations, so that with time the relevant characterizations become progressively more meaningful and accurate. Likewise, hurdles to broad general acceptance of any new clinical test tend to require more conclusive evidence than is needed to begin piloting select applications among early adopters. Furthermore, successful clinical validation may involve targeted investigating of correlations specifically exhibited with particular patient groups or conditions.

Clinical feasibility for any diagnostic or other applications is facilitated by performing fragility analyses with devices or systems capable of combining in a single integrated system stress application with measurement of induced hemolysis, as well as with results processing and output. For research uses and especially for clinical uses of MF testing—convenience, safety, and reliability can be substantially enhanced by employing a disposable single-use chip or cartridge component or the like to self-contain the blood sample during testing. A disposable component can allow several advantages for many of the above-noted applications of MF testing, depending upon the overall MF testing approach and configuration; usage of a replaceable disposable component for holding one or more blood samples to be tested during any given operation of the system can be more conducive to designing for concurrent or successive replication, for multiplexing either the dimensionality of fragility profiles and/or the number of distinct samples being tested at once while protecting against cross-contamination. It may also be more conducive to incorporating system calibration capabilities specific to a given configuration and/or kind of sample (e.g. pRBC), to further improve the reproducibility and reliability of test results.

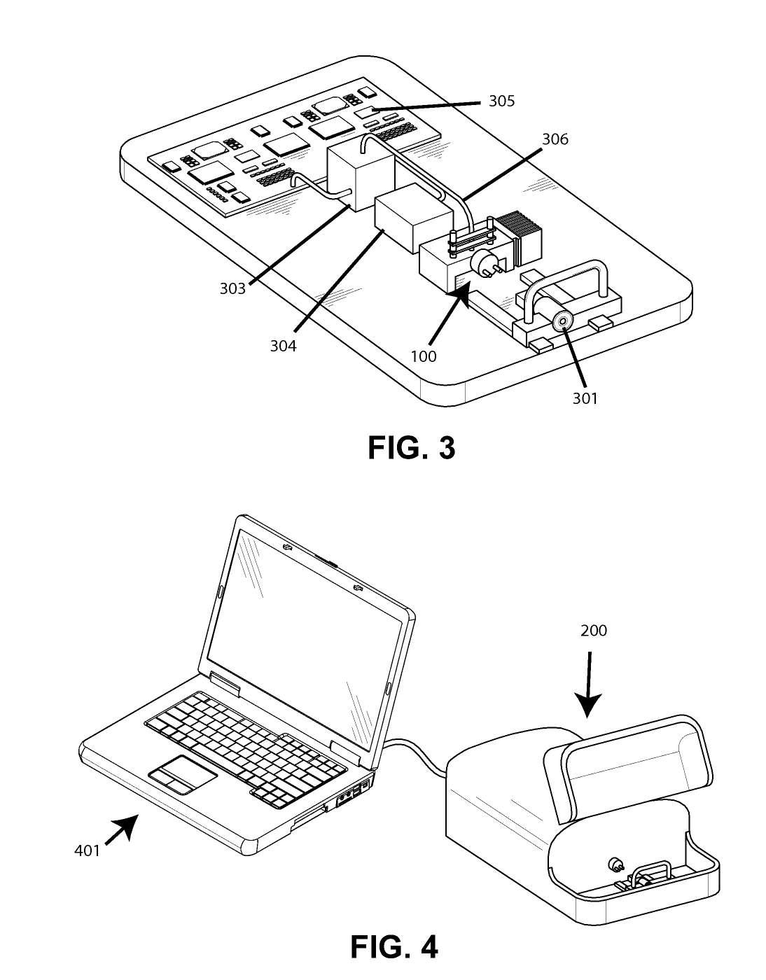

Various approaches for stressing and/or detection can be approached in conjunction with employing a single-use disposable component for containing the sample during the stressing (and preferably during the detection as well, albeit optionally in a different part thereof). In the context of a system directed toward blood product quality testing, for example, a rather generalized form of the disposable-utilizing approach is in claim 8 and in FIGS. 3 and 4 of U.S. Pat. No. 8,268,244, issued Sep. 18, 2012, which is herein incorporated by reference in its entirety, and which was a divisional of the original '916 application. Although that patent was primarily focused upon blood-banking/transfusion applications of fragility testing, that example (comprising a concentric-cylinder based stressor unit, whereby the unit includes a motor to turn one of the cylinders of the cartridge's lysing chamber, and with optical detection for determining induced hemolysis, e.g via spectral measurement) is more generally-applicable as well.

Ultrasound (also US, or U-S) via sonication is another one of the possible approaches for providing some or all of the mechanical stress in a MF assay. U-S involves multiple causes of its resulting hemolysis, which can be induced via fluidically-translated mechanical forces (a physical cause) and also via free radicals generated in the sample (a non-physical cause), the latter of which can be a confounding factor that is generally best to minimize in a fragility test so that “physical” causes will predominate.

For an U-S based fragility measurement to be accurate, the application of physical stress needs to be configured to ensure that other (non-physical) potential causes of hemolysis are not unduly affecting the results—which are supposed to primarily reflect susceptibility to applied physical stress, at least for uses where non-physical effects are not desired to be reflected in fragility results. In such cases, hemolysis from radicals should ideally be essentially negligible in comparison to hemolysis from physical effects. Hence, to usefully employ sonication for a fragility assay, it is important to ascertain the relative extent to which radicalization is responsible for the hemolysis. Existing literature indicates that generally the physical effects predominate over the chemical (radical) effects. Preliminary experiments conducted by the present inventors largely agree with this, and further indicate that this is mainly true for relatively low ultrasound intensities—the particular values for which vary of course by the particular configurations employed. These experiments were conducted with a bench-top sonicator and they compared RBC units with and without reagent for negating the effect of radicalization, in order to assess the relative roles of physical and chemical causes for ultrasound-induced hemolysis at different ultrasound intensities (power levels). Aside from power intensity and duration, other variable stress parameters could include for example sonication frequency as well as various solvent properties. The maximum desirable intensity threshold may need to be experimentally-determined for each geometry, volume, etc., and depend on whether direct or indirect contact is employed. (Note that this issue of U-S “intensity” as discussed here is not necessarily the same as the earlier-discussed issue of high-energy versus low-energy types of stress.)

Regarding the matter of cell concentration with a U-S based fragility test, existing literature establishes the general principle that cell concentration (hematocrit) is a factor for hemolysis efficiency. The inventors' preliminary experimentation largely agrees with this, and further indicated (through serial dilution experiments performed with a bench-top sonicator) that when the hematocrit is low enough (for a given ultrasonic stress intensity) this can essentially be neglected. (The particular threshold levels for dilution and associated sonication intensity may need to be determined experimentally for any given configuration.) Hence, either dilution should be normalized to establish a uniform hematocrit, or else dilution should be high enough to avoid the need for normalization.

Various other aspects of a US-based MF testing parameters tend to need to be empirically validated and optimized, some of which are configuration-specific and some of which might be fairly generalizable. For example, inventors' preliminary data suggests that “pulsing” vs. continuous application of ultrasound does not seem to make an appreciable difference so long as the cumulative duration of ultrasound application is the same and the temperature increase due to US power dissipating in the sonicated sample is ensured to not be significantly affecting the sample's properties (e.g. sample viscosity or RBC membrane fluidity). On the other hand, for example, sample volume is a significant factor in lysis efficiency and thus must always be accounted for.

Unlike with some other mechanical stressors, U-S stress does not necessarily involve the sample interacting principally with a solid object or rigid surface—a fact that makes sonication somewhat more conducive to occurring in the same chamber as detection. Yet this can optionally be changed, such as if combined with commercially-available micro-beads or the like, which may introduce a low-energy bead-induced stress in combination with the high-energy U-S stress. Note that if combining multiple different means or sources of stress together, their respective contributions to any results must be studied in order to assess their combined suitability for any given application. More generally, another benefit of employing disposable cartridges for the samples is the ability to provide different kinds of cartridges compatible with a given system—thereby allowing a change in the assays being run merely by changing the kind of cartridge being used. For example, some cartridges may be selectively offered with built-in micro-beads of varying sorts, different buffer solutions or internal dimensionalities, etc.

In an alternative set of embodiments of a related device or corresponding method, a sample can be split into distinct subsamples which respectively receive only a high-energy mechanical stress (for example, via a sonicator) and only a lower-energy mechanical stress (for example, via a conventional bead mill). Hemolysis can be tracked in each case to give a fragility profile for each respective kind of stress. Such joint profiling could then be used to produce a joint index whose value comprises some blend of the information obtained from the two profiles. As a simple example, one potential joint index could comprise an average of the durations of the two different stress kinds at given intensities that each corresponds to 50% hemolysis in their respective subsamples. This could be useful in cases where high-energy MF profiles and low-energy MF profiles may tend to differ substantially. For example, stored blood tends to experience increases in membrane rigidity as well as changes in shape; hence, this might be expected to result in degraded blood having a greater susceptibility to certain stressing means, while a lower susceptibility to others (which could be tracked over time during storage, by a joint index). Embodiments of this approach can optionally involve using consumable/disposable pieces also.

This disclosure now describes certain example approaches and embodiments for particular aspects of the invention (as depicted in the accompanying figures).

FIG. 1 shows a single-use disposable cartridge (i.e. sample-holding component) 100, structured in this case as a cuvette/cartridge that can serve as a combined chamber for contents undergoing stress, in this instance via sonication (administered via probe 101 and transferred via gasket 102), and also for taking optical readings via its compressible/flexible region 103 which in this case serves as both a single-chamber stressing section as well as a single-region detection section (above and below which fiber optics can close in to take readings at desired cycle intervals), and that region can be pinched between stress intervals to achieve an adequate spectrophotometric gap. It would be filled with blood via the sample port 104 before its insertion into the device. The sonication probes are disposable, and built into the cuvette. The detection window 105 is mounted on a film layer which enables the device to accurately capture a thin (predetermined height) layer of blood within the detectable portion of the cuvette at each detection cycle.

As shown next in FIGS. 2, 3, and 4, the consumable fits into a bench-top unit, which contains the necessary interfacing electronics and mechanical fixtures, which in turn plugs into a computer for software control. Specifically, FIG. 2 shows a single-sample cuvette 100 inserting into the base unit (benchtop/tabletop) device 200, with the sonication head 201 ready to be moved into place before the lid of the device is closed. FIG. 3 shows the interior of the base unit, where beneath the device shroud is the sonication driver 301 aligned with the cartridge 100 which is held in an alignment and cooling (temperature stabilization) fixture 302. A light source 303 and an optical analysis unit 304 are controlled from the control/analysis board 305 and can connect optically to the cartridge via fiber optics 306. FIG. 4 then shows the overall system for testing a single non-split sample, with supporting computer 401 for control and analysis, which could have instead optionally been incorporated directly into the base unit 200 of the test system. (It is possible that multiple single-sample base units could be connected to one USB hub and run from the same computer, to allow processing of multiple samples at the same time.)

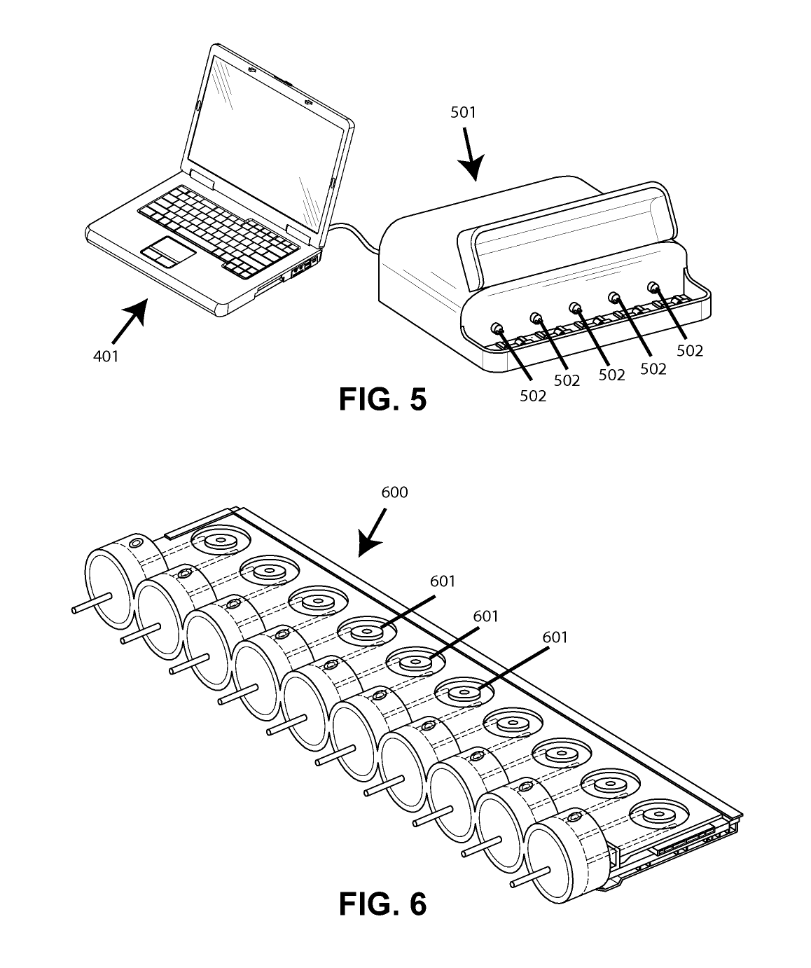

Alternatively, FIG. 5 shows how a multiplexed base unit 501 can accommodate multiple single-sample cartridges at once via parallel lanes 502. (This would be primarily a re-packaging of the single-lane base unit, as it would use the same single-lane cuvette for each blood sample or sub-sample to be tested.)

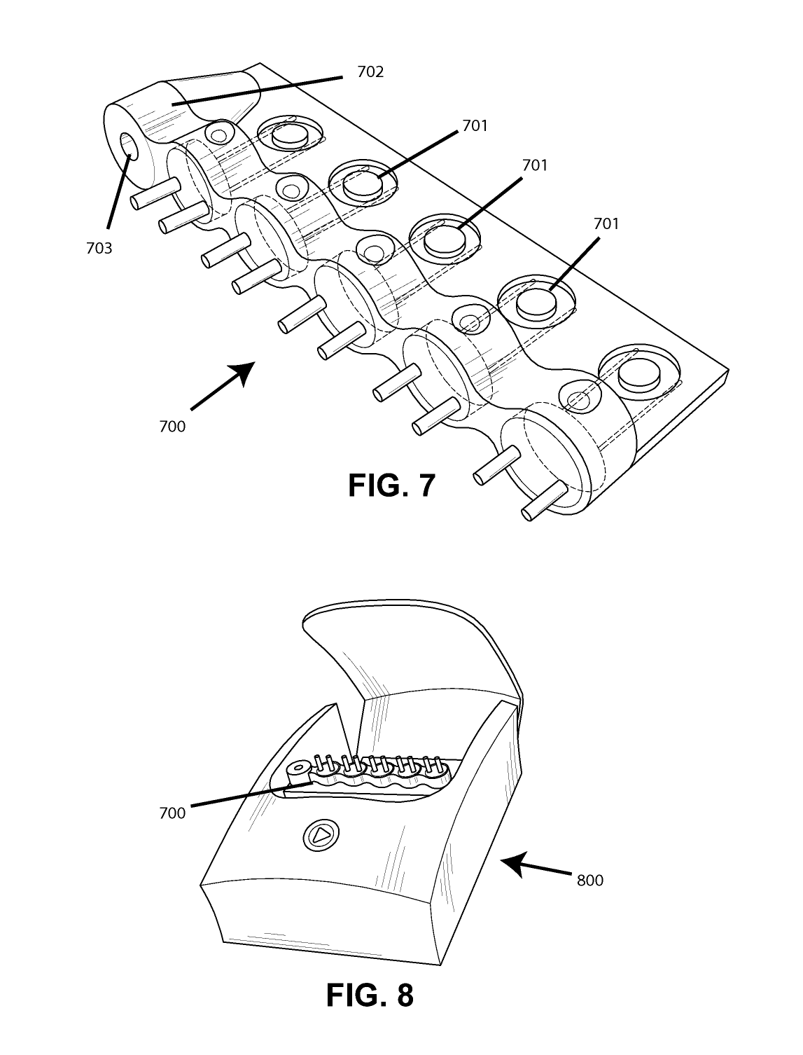

As further shown in FIGS. 6 and 7, the single chamber/cuvette concept from FIG. 1 can itself be replicated as a modular unit for multiplexing either the dimensionality of fragility profiles (e.g., via splitting a sample into subsamples going to multiple subsystems each providing a different stress intensity for a range of durations), and/or the kinds of fragility being tested (e.g., with and without beads), and/or the number of distinct samples being tested at once. Specifically, FIG. 6 depicts a multi-sample cartridge 600 consisting of linked cuvettes 601 (each of which serves here as both a stressing section and a detection section for each respective sample, akin to 103) for multiple distinct samples to be run simultaneously (each one as before), while FIG. 7 depicts a single-sample but multi-sub-sample cartridge 700 which aliquots a single blood sample into sub-samples (automatically) so that different stress/lysis conditions can be applied to each “sub-cuvette” or subsample-cuvette 701 (each of which serves here as both a stressing section and a detection section for each respective sub-sample of a given sample) to facilitate multidimensional fragility profiling of the sample. In FIG. 7, a common-receptacle 702 at one end takes a single sample through its port 703 and is linked to the bottom of each sub-cuvette such that the fluid level can equilibrate before insertion into the device. (Upon insertion, a membrane on the cartridge is sealed, trapping each sub-sample into its respective sub-cuvette and isolating it from the other sub-cuvettes.)

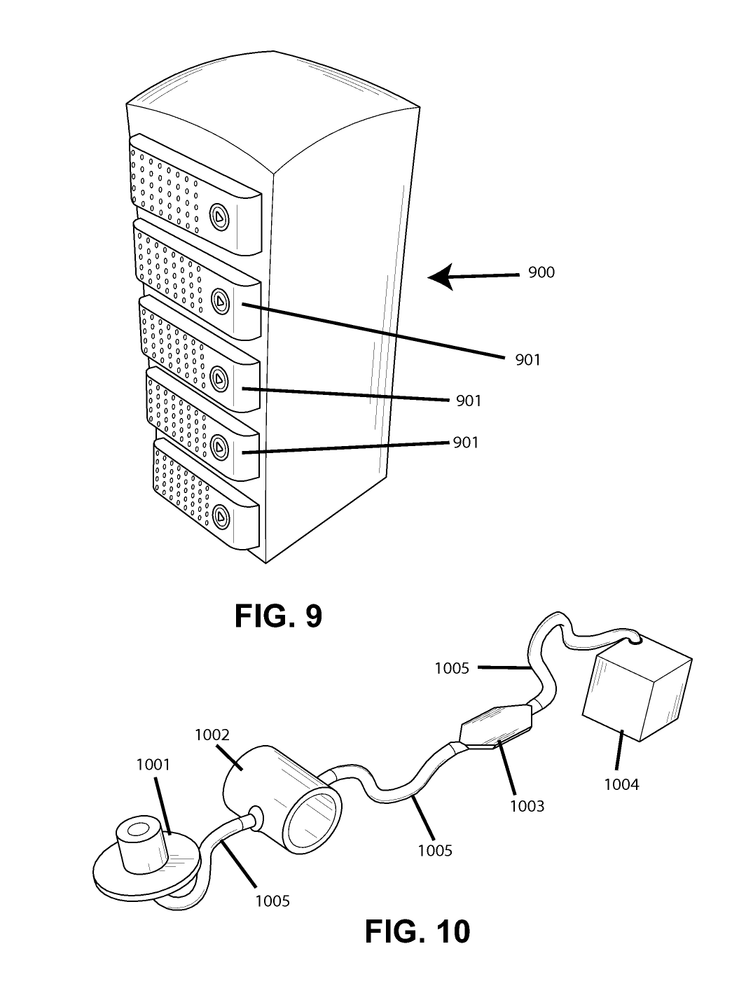

FIGS. 8 and 9 then depict a way to combine splitting a sample into subsamples (e.g. for multi-dimensional profiling of each sample) while also testing multiple different samples at once. Each distinct sample is given its own multi-compartment cartridge for splitting into subsamples, and placed in its own casing device, and then multiple such devices are then run in parallel by a single computer. As shown in FIG. 8, each single-sample/multi-sub-sample cartridge 700 can be inserted into a small casing device 800 containing the cooling, sonication, and optical features (not shown) for each sub-cuvette. FIG. 9 then shows a multi-cartridge tower 900 which would allow such cartridges in their respective casings to be inserted as tower “drawers” 901.

FIG. 10 shows an example of an embodiment of the single-use disposable cartridge (i.e., sample-holding component) having separate sections/portions configured for contents undergoing stress (in this instance via concentric cylinders, with sample residing in the gap while one of them rotates, the inherent effect of which is to cause cell-wall/cell-surface interaction and fluidic shear from sample flowing between the cylinders) and optical detection (in this instance via a thin rigid cuvette portion, to which sample flows from the stressing portion). A syringe dock with a sample entry port 1001 is connected to a concentric-cylinder-based lysis chamber (a single-chamber stressing section) 1002 via tubing 1005. The lysis chamber 1002 is connected to an optical cuvette (a single-region detection section) 1003 via tubing 1005, so that portions of sample can flow from the lysis chamber to the detection region/cuvette as respective intervals of stress are received. The optical cuvette 1003 is connected to a waste reservoir 1004 via tubing 1005.

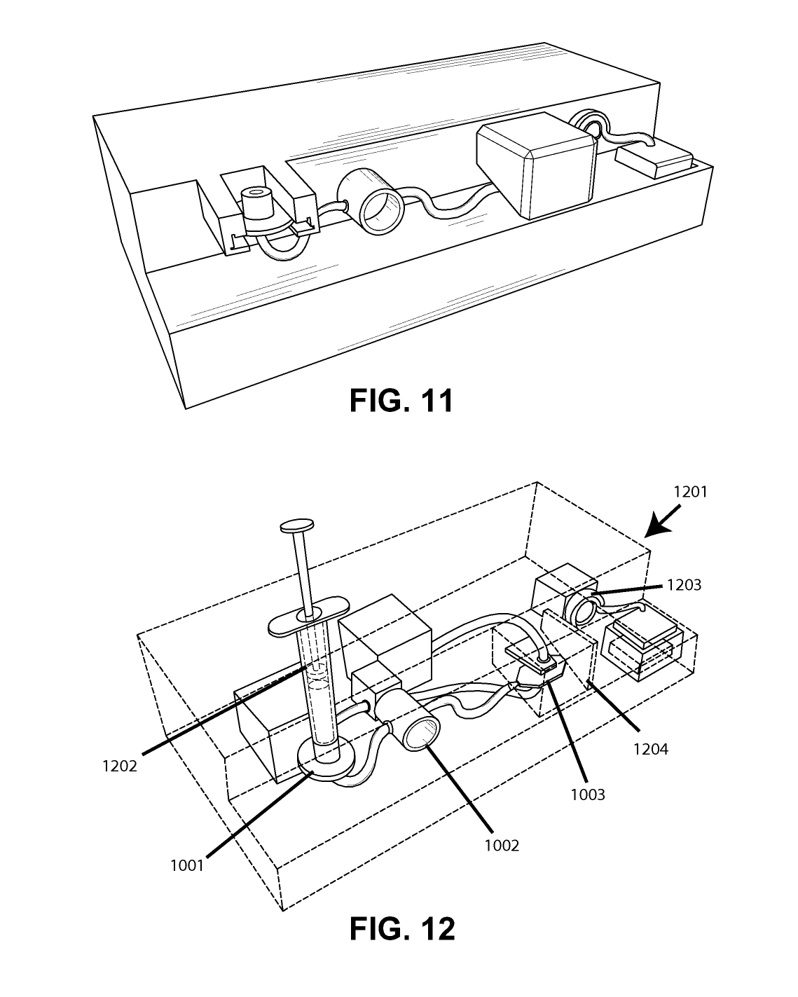

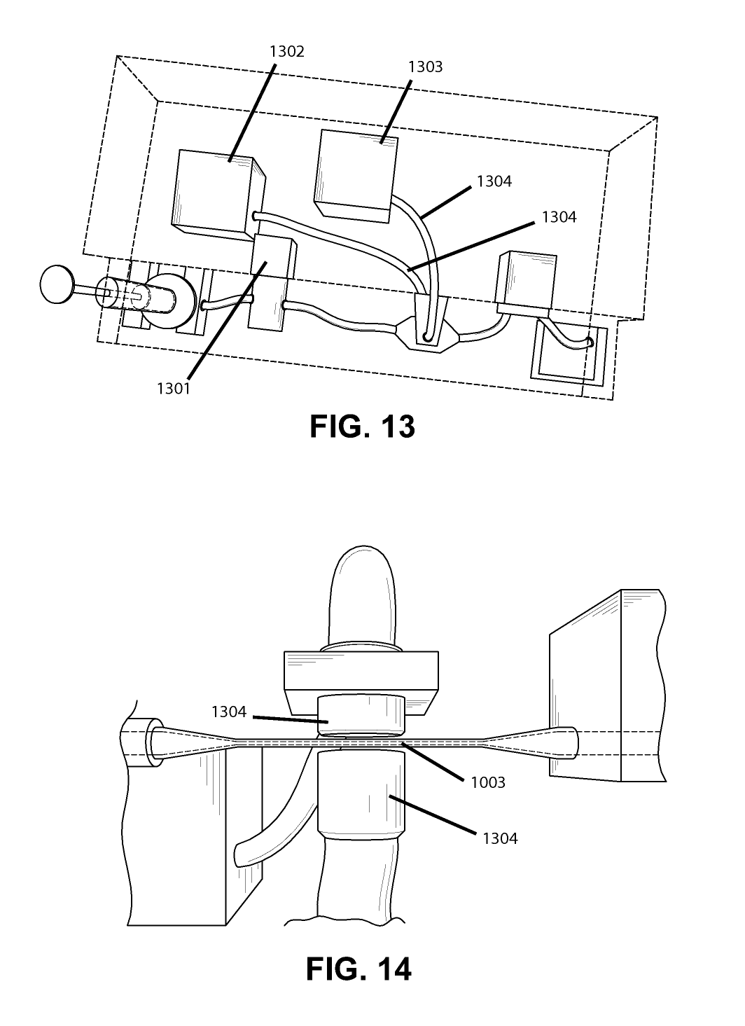

FIG. 11 shows a basic view of how the disposable from FIG. 10 fits into a corresponding base unit (benchtop/tabletop device) for a mechanical fragility test system, while FIGS. 12 and 13 show a see-through view of the device from the front and top, respectively, and with a door closed over the optical portion. A syringe 1202 injects a sample into the syringe dock 1001. An integrated peristaltic pump 1203 moves sample through the disposable component for processing by respective portions of the overall base unit 1201, such processing including cell lysis in the lysis chamber 1002 wherein an inner cylinder is turned by a built-in motor 1301, and also subsequent analysis of the sample in the optical cuvette portion 1003 occurring under a closed light-tight door 1204 via a spectrophotometer 1302 in conjunction with a light source 1303 which both employ fiber optic bundles 1304 to connect (optically) to the cuvette. FIG. 14 shows a frontal zoom of the optical detection portion, wherein said fiber optics 1304 sandwich said cuvette 1003.

Fixed or normalized dilution of cell concentrations can be performed either by the user or automated in the system. Alternatively, the consumable piece could be pre-filled with buffer from manufacture. Note that in the case of stored/packed RBC (pRBC), the storage solution for the main bag may be different from that of the test-segments (which could be a factor in whether such segments are deemed sufficiently representative of the main bag for a given purpose).

In an alternate embodiment applicable particularly for testing stored RBC in a bag, fragility measurement potentially could be performed without requiring a test sample being extracted. This could involve, for example, an optical hemolysis analysis component capable of directly measuring through bag material, which clamps or closes upon a portion of the bag which when in a clamped position contains only a small portion of the blood product (optionally, such cabined portions could remain sealed afterwards as well, depending upon the means used). In conjunction with this is also a stressor component, which subjects said small portion(s) of contents to stress while it remains in the bag; this could in effect make the bag itself a sort of a disposable container for any samples so cabined therein.

It's important to contrast any kind of RBC “fragility” with the related property of cell “deformability”—which is a broad concept covering many different kinds of tests that all in some way seek to determine how well a cell can deform or change shape under stress. Moreover, fragility (MF in particular) is particularly well suited for multi-parameter (>1) stressing to give multi-dimensional (>2) profiles showing how hemolysis depends on two or more stress variables such as extent/degree of intensity and extent/degree of duration (for one or more given type/kind) of mechanical stress; of course, any profiling based on one or more defined stress parameters implicitly presumes that any other conditions/variables affecting lysis are controlled, and as noted above sometimes normalization of hematocrit is needed as well. Indeed, merely providing the available option of such data richness can potentially enhance the general utility of embracing MF over other RBC membrane-related metrics.

The cartridge-based (or the like) system herein could also potentially be adapted to test mechanical fragility of material other than red blood cells. Of course the stressor(s)' selection and configuration would need to be empirically assessed and modified as appropriate to suit such alternative material, and for a type of cells or tissue other than red blood cells an appropriately modified spectral or cell-counting approach to detection of lysis/rupture would be needed.

This disclosure is enabling to those of ordinary skill in the art, while maintaining adequate flexibility for reasonable adaptation. Moreover, those skilled in the art will appreciate variations of the examples and principles described herein, which are also intended to be within the scope of the present invention. Any references herein to “the invention” or the like are thus intended in this spirit, as well as reflecting the respective subject matter of this particular filing. This section contains descriptive content for this disclosure. It is also to be understood that the terminology and phraseology used herein is for explanatory and exemplary purposes and not intended to be limiting, as the scope of the present invention is defined ultimately by the claims herein (as amended).

Erythrocyte mechanical fragility testing combines controlled physical (and more specifically, mechanical) stressing of cells along with a measurement of how much hemolysis occurs during such stressing. Most expressed definitions pertain to a notion of propensity for or susceptibility to hemolysis under mechanical stress. A wide range of research and clinical applications are possible for such metrics. Other (i.e., “non-mechanical”) kinds of fragility generally focus on other physical stresses—such as osmotic or photonic stress. Mechanical fragility has the potential to more relevantly (for select purposes) reflect erythrocyte membrane fragility, as osmotic fragility for example significantly depends on non-mechanical aspects such as intracellular ion concentration and transmembrane water/ion rates (in addition to mechanical properties). Sometimes the nomenclature or classification can vary, especially at the boundaries, or in cases of combinations or overlap. Likewise, within mechanical stress types, precise sub-categorization can vary as well (e.g. exact definitional limits or intersections of shear, pressure, stretch/tension, etc.).

There are many ways to provide the mechanical stress in a fragility assay, as well as many ways to measure the resulting hemolysis. As described by Tarssanen (1976), approaches to providing mechanical stress can be grouped into two broad categories: “high-energy” mechanical stress, and “low-energy” mechanical stress. Low-energy mechanical fragility, such as for example that utilizing a bead mill for the stress, tends to more directly reflect erythrocyte membrane properties, whereas high-energy mechanical fragility, such as that for example utilizing sonication for the stress, tends to more directly reflect hemoglobin viscosity and cell size. (Note that both general kinds of fragility assays could be useful for different purposes, and potentially for overlapping applications, and both kinds of stress are still “high” compared to that typically used for “deformability” measurements—in the sense of being potentially lethal to RBC and thus useful to hemolyze at least some of the cells as is needed for any “fragility” assay).

Certain kinds of mechanical stress, such as viscous stress, can be high-energy or low-energy; also, a fragility profile for some stress mechanisms may reflect membrane properties more directly in the lower ranges but reflect surface-to-volume ratio and viscosity more directly in the higher ranges. The implications of these differing kinds of effects can be that fragility tests employing different stress means and/or energy levels can correlate differently to a given phenomenon; to some extent this can be true even among different forms of low-energy stresses (e.g., bead mill vs. capillary tube). Just as osmotic and mechanical fragility can sometimes have inverse correlations to certain phenomena, likewise so can high-energy and low-energy variations of mechanical fragility. Also, sometimes stressors can be combined, such as when evaluating changes in mechanical fragility under differing osmolarities; relatedly, temperature or pressure or other factors can in some cases be relevant stress conditions.

However the stress gets provided for a given fragility test, there are also many ways to in effect measure the hemolysis resulting from the stress. Whenever the particular advantages of cell-counting (discussed in the noted Ser. No. 13/213,576 application) are not needed, the preferred approach is to use the Blood Hemolysis Analyzer disclosed in U.S. Pat. No. 7,790,464, issued Sep. 7, 2010, incorporated herein by reference in its entirety, which notably avoids the need for any separation steps to separate liquid from solid sample components (such as for example by centrifugation), as its innovation included exploiting a difference in “apparent” spectral absorption between extracellular/cell-free hemoglobin (Hb) and intracellular Hb, which in turn can be utilized to infer what proportion of a given sample is hemolysed. That patent notes that the desired sample thickness for its measurement can depend upon hematocrit, but in some embodiments may be between 0.05-0.5 mm. In general, among all possible means for detecting/measuring hemolysis level, optical ones are preferred; among optical means, spectral ones are preferred; among spectral means, those utilizing absorbance are preferred. As determining fractional hemolysis requires knowing the original (pre-lysis) hematocrit or a reasonable proxy therefor such as total hemoglobin concentration, two possible approaches for obtaining this piece of information are as follows: 1) achieving 100% hemolysis in at least one subsample as part of the stressing step, and/or 2) employing additionally the spectral analysis of blood in the visible spectral range (which can provide total Hb, which is the combined intracellular and extra-cellular hemoglobin).

A fragility assay can involve outputting various kinds or amounts of fragility data—including specific values or indices, single-variable-parameter fragility profiles, and/or multi-variable-parameter (“multi-dimensional”) fragility profiles. (Note that terms such as “parameter” can be used to refer to either a stress parameter—which can be varied to create a profile—or a fragility index value which may itself be based on or derived from such a profile, depending on the context.) Data matrices comprising how much lysis occurs under various combinations of stress parameters can be used to yield profile-based parameters characterizing the sample tested (or the source it represents). Data of interest (or inferable information therefrom) could comprise how much lysis would be expected under a given set of stress parameters, what stress condition(s) would be expected to result in a given lysis level, or slopes or shapes of any such curves/trajectories—the latter of which can reveal subpopulations within a sample with their own discernible profiles. The distribution of cell ages (i.e., physiological/metabolic ages, distinguished from blood product/unit ages) within a given person or animal may be a factor in sub-populations within a sample exhibiting distinct characteristics; nevertheless, often it is desirable for a single value (e.g., an average via some fragility-based metric) to represent an overall sample. The particular fragility parameter(s) sought will likely depend on what is deemed most clinically or scientifically relevant for each particular application. Notably, a single-value index parameter can itself be determined from a profile, such as for example through an interpolation (e.g., to estimate from available data points how much stress duration at a given intensity of a given type would be required to lyse a given percent of RBC in a given sample/source). Such fragility parameter(s) can be computed using a processor, which could be any kind of computer or like unit capable of generating desired output from raw measurements.

Depending on the stress/lysis method employed for a given mechanical fragility (MF) testing approach, it may be important to dilute samples to ensure that all samples (or subsamples) have the same concentration of red cells (hematocrit) in order to have consistency in the rate/efficiency of hemolysis when subjected to stress. Later herein it is addressed the extent to which this is an issue for a sonication-based stress/lysis method. Also, in the case of red cell samples experiencing aggregation or coagulation, it can be useful to ascertain the role of such on the cells' susceptibility to induced hemolysis.

Sensitivity with regard to spectrally-based measures of hemolysis can be enhanced by accounting for multiple forms of hemoglobin—namely oxy, deoxy, meth, and/or carboxy. It's uncommon to determine the concentration of all four types, and perhaps unnecessary for the amount of precision typically needed (including for the present invention), but nevertheless remains an option. Other absorbent proteins that may potentially interfere with hemoglobin measurements can be accounted for with multi-wavelength analysis.

Clinical applications of fragility-based red cell metrics can include blood product quality testing by measuring sub-lethal cell damage (e.g., extent, rate, acceleration, trajectory, etc.) existing during storage any time after donation due to aging, processing, storage conditions, etc. Alternatively, fragility (osmotic and/or mechanical) can be useful in various patient diagnostics, including for example diagnosing conditions caused wholly or partially by drug and/or device based medical therapies—as well as any number of pathological diseases known or found to have an effect upon red cell membrane properties.

Regarding the blood-banking/transfusion-medicine (or TM) related uses, the related original application Ser. No. 12/690,916 described previously-unexplored approaches for using in vitro testing of RBC membrane properties to reflect blood quality loss in a time-independent manner; in particular, it suggested using RBC fragility, preferably mechanical, to indicate stored blood quality or loss thereof, and it notably presented preliminary in vitro data showing that RBC units of the same age can differ substantially in their mechanical fragility profiles. U.S. Pat. No. 8,263,408, issued Sep. 11, 2012 (resulting from a related divisional application of the noted parent) is also herein incorporated by reference and addresses “red blood cell suitability for transfusion,” tests for which can involve either donor (i.e. directly drawn from a donor or prospective donor) or donated (i.e. stored) red cells.

An extension of the blood product quality application can be to evaluate effects of blood product collection manner (e.g. apheresis vs. whole-blood) or manufacturing processes (e.g. leukoreduction, irradiation); or to validate associated processing equipment, as well as any storage/handling/transportation materials or conditions (e.g. bag material, storage or rejuvenation solutions, temperature ranges), by examining their respective effects upon blood quality or transfusion suitability (in terms of RBC fragility). Such RBC membrane properties can also be used to ascertain beforehand which units may be most amenable to certain manufacturing processes or storage conditions, by establishing a predictive correlation (directly or indirectly) between the in vitro property before subjection to said process or condition and relative in vivo performance. As with other applications, particular fragility assays or parameters may prove useful in combination with each other and/or other assays (e.g. biochemical).

Ongoing research in TM will progressively strengthen the case for clinical adoption of the MF test, with each round of studies likely enticing additional users for more use contexts (clinical opinion varies as to which kinds of studies would be most conclusive, as well as which applications would be most useful). Measurable degrees of quality/suitability could be used to reflect degrees of product acceptability, such as by selectively triaging those units deemed to be the “best” or least-degraded toward the most vulnerable patients (or for those unit transfusions where oxygenation is deemed the most critical, etc.). Moreover, the multi-parametric/multi-dimensional potential of MF could enable different “kinds” of quality to be discerned which may respectively prove more or less clinically relevant for different patients and/or patient groups/conditions. For example, post-transfusion in vivo RBC survival and RBC perfusion may each tend to be more associated with distinct respective multi-dimensional profile characteristics, as may resultant tissue oxygenation. Particular profile-based fragility parameter(s) or index(es) may thus prove indicative of RBC ability to adapt to the physical and/or chemical environment within the patient/recipient in a way that varies inter-donor, intra-donor, and even intra-unit (e.g., via sub-populations or cell-by-cell differences).

In effect, storage time has generally been the principal indicator of blood quality loss as applicable to particular individual units—although notably, the usefulness of even this indicator remains the subject of much controversy. This highlights the value of establishing a unit-specific RBC quality test to replace or supplement mere storage time as an indicator of storage lesion—as well as rates thereof, by testing at multiple points in time—thereby allowing modifications in today's inventory management practices (presently dominated by “first-in-first-out,” or FIFO). And in some cases, the RBC membrane properties of a patient's own blood may also be a factor in what kind of RBC properties are needed from stored blood they would receive in a transfusion. Variations among RBC units right at donation/collection can be at least partly attributable to “donor-to-donor” differences, which can also be reflected in subsequent degradation during storage (along with other factors). Dern et al. had first showed inter-donor variation in RBC “storageability” as measured by post-transfusion survival in vivo, and looked at some possible in vitro metrics (both membrane-related and strictly biochemical); Card et al. subsequently showed related findings (regarding inter-donor differences in certain RBC membrane properties). In some cases such differences may result from some identifiable medical condition, but there can also be a substantial range of variation among “healthy” donors. Also notable is that even “same-donor” differences can exist from one donation to the next.

In other (non-TM) applications, mechanical fragility (MF) can be used to evaluate or validate various blood-handling devices (or medical procedures employing them) by measuring sub-lethal RBC injury they cause manifesting in higher MF (e.g. Kamaneva et al. 2002, Yazer et al. 2008); relatedly, MF testing can be used to calibrate or account for MF to facilitate a consistent standard of evaluation of the actual hemolysis caused by blood-handling devices (e.g. Gu et al. 2005). Note also that evaluation of blood-handling devices via their effects on patients can be distinguished from actual patient diagnosis of a condition for which such a device may in fact be a cause.

There still as yet is no well-established or universally-standardized means to test for RBC MF—a fact that may have limited its utility for any of the above-noted or other applications. Of course, a vital aspect of optimal implementation for any application of any new life-science test includes the accumulation of copious output and associated correlations, so that with time the relevant characterizations become progressively more meaningful and accurate. Likewise, hurdles to broad general acceptance of any new clinical test tend to require more conclusive evidence than is needed to begin piloting select applications among early adopters. Furthermore, successful clinical validation may involve targeted investigating of correlations specifically exhibited with particular patient groups or conditions.

Clinical feasibility for any diagnostic or other applications is facilitated by performing fragility analyses with devices or systems capable of combining in a single integrated system stress application with measurement of induced hemolysis, as well as with results processing and output. For research uses and especially for clinical uses of MF testing—convenience, safety, and reliability can be substantially enhanced by employing a disposable single-use chip or cartridge component or the like to self-contain the blood sample during testing. A disposable component can allow several advantages for many of the above-noted applications of MF testing, depending upon the overall MF testing approach and configuration; usage of a replaceable disposable component for holding one or more blood samples to be tested during any given operation of the system can be more conducive to designing for concurrent or successive replication, for multiplexing either the dimensionality of fragility profiles and/or the number of distinct samples being tested at once while protecting against cross-contamination. It may also be more conducive to incorporating system calibration capabilities specific to a given configuration and/or kind of sample (e.g. pRBC), to further improve the reproducibility and reliability of test results.

Various approaches for stressing and/or detection can be approached in conjunction with employing a single-use disposable component for containing the sample during the stressing (and preferably during the detection as well, albeit optionally in a different part thereof). In the context of a system directed toward blood product quality testing, for example, a rather generalized form of the disposable-utilizing approach is in claim 8 and in FIGS. 3 and 4 of U.S. Pat. No. 8,268,244, issued Sep. 18, 2012, which is herein incorporated by reference in its entirety, and which was a divisional of the original '916 application. Although that patent was primarily focused upon blood-banking/transfusion applications of fragility testing, that example (comprising a concentric-cylinder based stressor unit, whereby the unit includes a motor to turn one of the cylinders of the cartridge's lysing chamber, and with optical detection for determining induced hemolysis, e.g via spectral measurement) is more generally-applicable as well.

Ultrasound (also US, or U-S) via sonication is another one of the possible approaches for providing some or all of the mechanical stress in a MF assay. U-S involves multiple causes of its resulting hemolysis, which can be induced via fluidically-translated mechanical forces (a physical cause) and also via free radicals generated in the sample (a non-physical cause), the latter of which can be a confounding factor that is generally best to minimize in a fragility test so that “physical” causes will predominate.

For an U-S based fragility measurement to be accurate, the application of physical stress needs to be configured to ensure that other (non-physical) potential causes of hemolysis are not unduly affecting the results—which are supposed to primarily reflect susceptibility to applied physical stress, at least for uses where non-physical effects are not desired to be reflected in fragility results. In such cases, hemolysis from radicals should ideally be essentially negligible in comparison to hemolysis from physical effects. Hence, to usefully employ sonication for a fragility assay, it is important to ascertain the relative extent to which radicalization is responsible for the hemolysis. Existing literature indicates that generally the physical effects predominate over the chemical (radical) effects. Preliminary experiments conducted by the present inventors largely agree with this, and further indicate that this is mainly true for relatively low ultrasound intensities—the particular values for which vary of course by the particular configurations employed. These experiments were conducted with a bench-top sonicator and they compared RBC units with and without reagent for negating the effect of radicalization, in order to assess the relative roles of physical and chemical causes for ultrasound-induced hemolysis at different ultrasound intensities (power levels). Aside from power intensity and duration, other variable stress parameters could include for example sonication frequency as well as various solvent properties. The maximum desirable intensity threshold may need to be experimentally-determined for each geometry, volume, etc., and depend on whether direct or indirect contact is employed. (Note that this issue of U-S “intensity” as discussed here is not necessarily the same as the earlier-discussed issue of high-energy versus low-energy types of stress.)

Regarding the matter of cell concentration with a U-S based fragility test, existing literature establishes the general principle that cell concentration (hematocrit) is a factor for hemolysis efficiency. The inventors' preliminary experimentation largely agrees with this, and further indicated (through serial dilution experiments performed with a bench-top sonicator) that when the hematocrit is low enough (for a given ultrasonic stress intensity) this can essentially be neglected. (The particular threshold levels for dilution and associated sonication intensity may need to be determined experimentally for any given configuration.) Hence, either dilution should be normalized to establish a uniform hematocrit, or else dilution should be high enough to avoid the need for normalization.

Various other aspects of a US-based MF testing parameters tend to need to be empirically validated and optimized, some of which are configuration-specific and some of which might be fairly generalizable. For example, inventors' preliminary data suggests that “pulsing” vs. continuous application of ultrasound does not seem to make an appreciable difference so long as the cumulative duration of ultrasound application is the same and the temperature increase due to US power dissipating in the sonicated sample is ensured to not be significantly affecting the sample's properties (e.g. sample viscosity or RBC membrane fluidity). On the other hand, for example, sample volume is a significant factor in lysis efficiency and thus must always be accounted for.

Unlike with some other mechanical stressors, U-S stress does not necessarily involve the sample interacting principally with a solid object or rigid surface—a fact that makes sonication somewhat more conducive to occurring in the same chamber as detection. Yet this can optionally be changed, such as if combined with commercially-available micro-beads or the like, which may introduce a low-energy bead-induced stress in combination with the high-energy U-S stress. Note that if combining multiple different means or sources of stress together, their respective contributions to any results must be studied in order to assess their combined suitability for any given application. More generally, another benefit of employing disposable cartridges for the samples is the ability to provide different kinds of cartridges compatible with a given system—thereby allowing a change in the assays being run merely by changing the kind of cartridge being used. For example, some cartridges may be selectively offered with built-in micro-beads of varying sorts, different buffer solutions or internal dimensionalities, etc.

In an alternative set of embodiments of a related device or corresponding method, a sample can be split into distinct subsamples which respectively receive only a high-energy mechanical stress (for example, via a sonicator) and only a lower-energy mechanical stress (for example, via a conventional bead mill). Hemolysis can be tracked in each case to give a fragility profile for each respective kind of stress. Such joint profiling could then be used to produce a joint index whose value comprises some blend of the information obtained from the two profiles. As a simple example, one potential joint index could comprise an average of the durations of the two different stress kinds at given intensities that each corresponds to 50% hemolysis in their respective subsamples. This could be useful in cases where high-energy MF profiles and low-energy MF profiles may tend to differ substantially. For example, stored blood tends to experience increases in membrane rigidity as well as changes in shape; hence, this might be expected to result in degraded blood having a greater susceptibility to certain stressing means, while a lower susceptibility to others (which could be tracked over time during storage, by a joint index). Embodiments of this approach can optionally involve using consumable/disposable pieces also.

This disclosure now describes certain example approaches and embodiments for particular aspects of the invention (as depicted in the accompanying figures).

FIG. 1 shows a single-use disposable cartridge (i.e. sample-holding component) 100, structured in this case as a cuvette/cartridge that can serve as a combined chamber for contents undergoing stress, in this instance via sonication (administered via probe 101 and transferred via gasket 102), and also for taking optical readings via its compressible/flexible region 103 which in this case serves as both a single-chamber stressing section as well as a single-region detection section (above and below which fiber optics can close in to take readings at desired cycle intervals), and that region can be pinched between stress intervals to achieve an adequate spectrophotometric gap. It would be filled with blood via the sample port 104 before its insertion into the device. The sonication probes are disposable, and built into the cuvette. The detection window 105 is mounted on a film layer which enables the device to accurately capture a thin (predetermined height) layer of blood within the detectable portion of the cuvette at each detection cycle.

As shown next in FIGS. 2, 3, and 4, the consumable fits into a bench-top unit, which contains the necessary interfacing electronics and mechanical fixtures, which in turn plugs into a computer for software control. Specifically, FIG. 2 shows a single-sample cuvette 100 inserting into the base unit (benchtop/tabletop) device 200, with the sonication head 201 ready to be moved into place before the lid of the device is closed. FIG. 3 shows the interior of the base unit, where beneath the device shroud is the sonication driver 301 aligned with the cartridge 100 which is held in an alignment and cooling (temperature stabilization) fixture 302. A light source 303 and an optical analysis unit 304 are controlled from the control/analysis board 305 and can connect optically to the cartridge via fiber optics 306. FIG. 4 then shows the overall system for testing a single non-split sample, with supporting computer 401 for control and analysis, which could have instead optionally been incorporated directly into the base unit 200 of the test system. (It is possible that multiple single-sample base units could be connected to one USB hub and run from the same computer, to allow processing of multiple samples at the same time.)

Alternatively, FIG. 5 shows how a multiplexed base unit 501 can accommodate multiple single-sample cartridges at once via parallel lanes 502. (This would be primarily a re-packaging of the single-lane base unit, as it would use the same single-lane cuvette for each blood sample or sub-sample to be tested.)

As further shown in FIGS. 6 and 7, the single chamber/cuvette concept from FIG. 1 can itself be replicated as a modular unit for multiplexing either the dimensionality of fragility profiles (e.g., via splitting a sample into subsamples going to multiple subsystems each providing a different stress intensity for a range of durations), and/or the kinds of fragility being tested (e.g., with and without beads), and/or the number of distinct samples being tested at once. Specifically, FIG. 6 depicts a multi-sample cartridge 600 consisting of linked cuvettes 601 (each of which serves here as both a stressing section and a detection section for each respective sample, akin to 103) for multiple distinct samples to be run simultaneously (each one as before), while FIG. 7 depicts a single-sample but multi-sub-sample cartridge 700 which aliquots a single blood sample into sub-samples (automatically) so that different stress/lysis conditions can be applied to each “sub-cuvette” or subsample-cuvette 701 (each of which serves here as both a stressing section and a detection section for each respective sub-sample of a given sample) to facilitate multidimensional fragility profiling of the sample. In FIG. 7, a common-receptacle 702 at one end takes a single sample through its port 703 and is linked to the bottom of each sub-cuvette such that the fluid level can equilibrate before insertion into the device. (Upon insertion, a membrane on the cartridge is sealed, trapping each sub-sample into its respective sub-cuvette and isolating it from the other sub-cuvettes.)

FIGS. 8 and 9 then depict a way to combine splitting a sample into subsamples (e.g. for multi-dimensional profiling of each sample) while also testing multiple different samples at once. Each distinct sample is given its own multi-compartment cartridge for splitting into subsamples, and placed in its own casing device, and then multiple such devices are then run in parallel by a single computer. As shown in FIG. 8, each single-sample/multi-sub-sample cartridge 700 can be inserted into a small casing device 800 containing the cooling, sonication, and optical features (not shown) for each sub-cuvette. FIG. 9 then shows a multi-cartridge tower 900 which would allow such cartridges in their respective casings to be inserted as tower “drawers” 901.

FIG. 10 shows an example of an embodiment of the single-use disposable cartridge (i.e., sample-holding component) having separate sections/portions configured for contents undergoing stress (in this instance via concentric cylinders, with sample residing in the gap while one of them rotates, thus causing cell-wall interaction and fluid shear) and optical detection (in this instance via a thin rigid cuvette portion, to which sample flows from the stressing portion). A syringe dock with a sample entry port 1001 is connected to a concentric-cylinder-based lysis chamber (a single-chamber stressing section) 1002 via tubing 1005. The lysis chamber 1002 is connected to an optical cuvette (a single-region detection section) 1003 via tubing 1005, so that portions of sample can flow from the lysis chamber to the detection region/cuvette as respective intervals of stress are received. The optical cuvette 1003 is connected to a waste reservoir 1004 via tubing 1005.

FIG. 11 shows a basic view of how the disposable from FIG. 10 fits into a corresponding base unit (benchtop/tabletop device) for a mechanical fragility test system, while FIGS. 12 and 13 show a see-through view of the device from the front and top, respectively, and with a door closed over the optical portion. A syringe 1202 injects a sample into the syringe dock 1001. An integrated peristaltic pump 1203 moves sample through the disposable component for processing by respective portions of the overall base unit 1201, such processing including cell lysis in the lysis chamber 1002 wherein an inner cylinder is turned by a built-in motor 1301, and also subsequent analysis of the sample in the optical cuvette portion 1003 occurring under a closed light-tight door 1204 via a spectrophotometer 1302 in conjunction with a light source 1303 which both employ fiber optic bundles 1304 to connect (optically) to the cuvette. FIG. 14 shows a frontal zoom of the optical detection portion, wherein said fiber optics 1304 sandwich said cuvette 1003.

Fixed or normalized dilution of cell concentrations can be performed either by the user or automated in the system. Alternatively, the consumable piece could be pre-filled with buffer from manufacture. Note that in the case of stored/packed RBC (pRBC), the storage solution for the main bag may be different from that of the test-segments (which could be a factor in whether such segments are deemed sufficiently representative of the main bag for a given purpose).

In an alternate embodiment applicable particularly for testing stored RBC in a bag, fragility measurement potentially could be performed without requiring a test sample being extracted. This could involve, for example, an optical hemolysis analysis component capable of directly measuring through bag material, which clamps or closes upon a portion of the bag which when in a clamped position contains only a small portion of the blood product (optionally, such cabined portions could remain sealed afterwards as well, depending upon the means used). In conjunction with this is also a stressor component, which subjects said small portion(s) of contents to stress while it remains in the bag; this could in effect make the bag itself a sort of a disposable container for any samples so cabined therein.

It's important to contrast any kind of RBC “fragility” with the related property of cell “deformability”—which is a broad concept covering many different kinds of tests that all in some way seek to determine how well a cell can deform or change shape under stress. Moreover, fragility (MF in particular) is particularly well suited for multi-parameter (>1) stressing to give multi-dimensional (>2) profiles showing how hemolysis depends on two or more stress variables such as extent/degree of intensity and extent/degree of duration (for one or more given type/kind) of mechanical stress. Indeed, merely providing the available option of such data richness can potentially enhance the general utility of embracing MF over other RBC membrane-related metrics.

The cartridge-based (or the like) system herein could also potentially be adapted to test mechanical fragility of material other than red blood cells. Of course the stressor(s)' selection and configuration would need to be empirically assessed and modified as appropriate to suit such alternative material, and for a type of cells or tissue other than red blood cells an appropriately modified spectral or cell-counting approach to detection of lysis/rupture would be needed.

This disclosure is enabling to those of ordinary skill in the art, while maintaining adequate flexibility for reasonable adaptation. Moreover, those skilled in the art will appreciate variations of the examples and principles described herein, which are also intended to be within the scope of the present invention. Any references herein to “the invention” or the like are thus intended in this spirit, as well as reflecting the respective subject matter of this particular filing.

We claim:

1. A disposable component configured to contain a sample(s) of red blood cells being profiled by a device for testing erythrocyte membrane mechanical fragility, the disposable component comprising:
a port(s) for receiving the sample(s);
a stressing section comprising a chamber(s) in which the sample(s) can receive intervals of mechanical stress from the device, to cause lysis in the sample(s) while the sample(s) remains within said chamber(s), such that the device does not contact the sample(s) while the sample(s) receives said stress, thereby preventing contamination of the device; and
a detection section comprising an optically transparent region(s) that at least sometimes has a thickness suitable for taking optical readings to reflect what proportion of the cells are lysed after each of said intervals of mechanical stress, with said region(s) having an area sufficient for said readings, whereby said region(s) can capture at least a portion(s) of the sample(s) that received said stress in said chamber(s), said portion(s) being sufficiently representative of said sample(s) such that said readings can reflect said lysis caused by said stress.

2. The component of claim 1, wherein said component can house two or more subsamples of one or more of said sample(s), and each of said subsamples can receive stress of a different intensity, whereby said sample(s) is able to be profiled three-dimensionally by plotting hemolysis versus stress intensity and stress duration.

3. The component of claim 1, wherein said component can house two or more of said sample(s), and each of said sample(s) can receive stress of the same intensity, whereby each of said sample(s) is able to be profiled two-dimensionally by plotting hemolysis versus stress duration.

4. The component of claim 1, in a configuration comprising at least one additional component of claim 1, such that two or more of said sample(s) can each be divided into two or more subsamples, and each of said subsamples from each of said sample(s) can receive stress of a different intensity, whereby each of said sample(s) is able to be profiled three-dimensionally by plotting hemolysis versus stress intensity and stress duration.

5. The component of claim 1, wherein said region(s) is a compressible region(s) which is compressible to temporarily and repeatedly achieve said thickness.

6. The component of claim 5, wherein the thickness is a predetermined thickness.

7. The component of claim 5, wherein the stressing section is configured for receiving stress from a sonicator.

8. The component of claim 1, wherein said stressing section and said detection section spatially coincide such that said chamber(s) can be reversibly pinched or compressed to allow said region(s) to be said thickness while capturing said portion(s) of said sample(s), thereby facilitating said readings.

9. The component of claim 8, wherein said thickness is a predetermined thickness.

10. The component of claim 8, wherein the component is configured for the sample to receive stress from a sonicator.

11. The component of claim 1, wherein there are multiple stressing sections and/or multiple detection sections.

12. The component of claim 1, further comprising additional stressing sections that are loadable by the same port, such that each stressing section receives a subsample of a given sample.

13. The component of claim 1, wherein said stressing section and said detection section are separate sections.

14. The component of claim 13, wherein said region(s) is rigid and said thickness is fixed.

15. The component of claim 13, wherein the stressing section is configured to allow cells in the sample(s) to experience stress from flow in the stressing section caused by said device.

16. The component of claim 8 or 13, wherein the optical readings are spectral readings.

17. The component of claim 8 or 13, wherein the optical readings comprise absorbance.

18. The component of claim 1, wherein the optical readings comprise spectral absorbance readings that indicate what proportion of hemoglobin is cell-free relative to total hemoglobin, thereby allowing an inference of proportional hemolysis without separation of solid and liquid sample portions.
